(19) United States
(12) Patent Application Publication
(10) Pub. No.: US 2026/0216020 A1
(43) Pub. Date: Jul. 30, 2026

(54) A COMPOSITION

(71) Applicant: Conopco, Inc., d/b/a UNILEVER, Hoboken, NJ (US)

(72) Inventors: Ranjit Kaur BHOGAL, Huntingdon (GB); Louise Jannette ROBERTS, Ellesmere Port (GB)

(73) Assignee: Conopco, Inc., d/b/a UNILEVER, Hoboken, NJ (US)

(21) Appl. No.: 19/145,683

(22) PCT Filed: Jan. 3, 2024
(86) PCT No.: PCT/EP2024/050073

§ 371 Date: Jul. 3, 2025

(30) Foreign Application Priority Data
Jan. 3, 2023 (EP) 23150222.0

(51) Int. Cl. A61K 8/34; A61K 8/60; A61K 8/73; A61K 8/92; A61Q 5/12

(52) U.S. Cl. CPC A61K 8/342; A61K 8/604; A61K 8/73; A61K 8/922; A61Q 5/12

(57) ABSTRACT

A cosmetic composition comprising: i) xanthan gum; ii) a combination of a C₈ to C₁₄ alcohol and C₈ to C₁₄ glucoside; and iiii) coconut oil; wherein the weight ratio of ii) a combination of a C₈ to C₁₄ alcohol and C₈ to C₁₄ glucoside and iii) coconut oil is 1:4 to 4:1.

### FIELD OF THE INVENTION

The present invention relates to personal care compositions in particular to hair conditioning compositions.

### BACKGROUND OF THE INVENTION

The present invention is directed to a composition that penetrates the stratum corneum. More particularly, the invention is directed to a prolipid composition that provides personalized benefits by rapidly restoring the skin's/scalps natural lipid balance. Such a composition results in elongated fatty acid and/or lipid production deep in the stratum corneum and near the surface of the stratum lucidum.

International patent application published as WO 02/067880 (Unilever) suggests an alternative approach to the treatment of dandruff. In particular WO 02/067880 discloses use of a lipophilic agent or a lipid precursor and/or promoter in the manufacture of a composition for treating and/or preventing dandruff by strengthening the scalp.

It is of increasing interest to develop a topical care (i.e., prolipid) composition that replenishes the skin's/scalps fatty acids and natural lipid matrix deep within the stratum corneum in order to produce a long lasting skin resilience barrier that is suitable to improve delivery to hair and skin of benefit agents while simultaneously sealing the scalp/skin for long lasting moisturization.

WO 22/136357 discloses prolipid composition for personalized benefits and method for using the same.

However, formulation of such compositions into products that are acceptable to the consumer can be difficult as in some instances the product can appear grainy, the product can have an unacceptable viscosity or with certain fatty acids the composition can be unstable.

### DESCRIPTION OF THE INVENTION

The present invention relates to a cosmetic composition comprising:
i) xanthan gum;
ii) a combination of a C₈ to C₁₄ alcohol and C₈ to C₁₄ glucoside; and
iiii) coconut oil
   wherein the weight ratio of ii) a combination of a C₈ to C₁₄ alcohol and C₈ to C₁₄ glucoside and iii) coconut oil is 1:4 to 4:1.

The present invention also relates to a method of conditioning the hair and scalp comprising the step of applying to the hair and scalp the composition according to any preceding claim.

### DETAILED DESCRIPTION OF THE INVENTION

Preferably, the cosmetic composition is a hair care composition.

According to a preferred aspect, the present invention provides a composition for conditioning the scalp against the onset, return or worsening of symptoms associated with dandruff. Such conditioning is preferably non-therapeutic, more preferably cosmetic.

By the term “strengthen the scalp”, and related terms used herein, we mean that the resistance of the scalp to penetration by moisture is increased i.e., the water permeability barrier of the scalp is improved and/or strengthened. The loss of water through the stratum corneum may also be inhibited. Alternative descriptions for the strengthening of the scalp barrier, which are encompassed by the term as used herein, include, but are not limited to, one or more of the following: scalp nourishment, restoring the scalp barrier, strengthening the scalp, strengthening scalp resistance/defense, helping scalp regenerate, rebuilding the scalp, making smoother scalp skin, soothing the scalp, replenishing the scalp from within, building the scalp, building the scalp from within, building the scalp barrier from within, revitalising the scalp, moisturising the scalp, fully hydrating the scalp, activating the scalp's natural protection layer, awakening scalp's natural protection layer and similar terms.

By strengthening the scalp, the scalp is in a better condition to resist the action of irritants such as Malassezia and/or microbial metabolites. Additionally, or alternatively the scalp may be in a better condition to resist degradation by harsh surfactants.

Thus in a preferred embodiment the scalp is conditioned against the onset, return or worsening of symptoms associated with dandruff following contact of the scalp with cleansing surfactant, especially anionic cleansing surfactant.

A preferred combination of a C₈ to C₁₄ alcohol and C₈ to C₁₄ glucoside is sold as Montanov S. Preferably the level C₈ to C₁₄ alcohol and C₈ to C₁₄ glucoside C₁₄ glucoside combination is from 0.5 to 7 wt %, more preferably from 1 to 5 wt % of the total composition.

The composition comprises coconut oil, (palmitic/stearic acid) as a scalp conditioner. Preferably the coconut oil is present from 0.5 to 10 wt %, more preferably from 2 to 7 wt % of the total composition.

In some compositions it is preferable if a hydroxy stearic acid, preferably 12-hydroxy stearic acid is present.

The weight ratio of ii) a combination of a C₈ to C₁₄ alcohol and C₈ to C₁₄ glucoside and iii) coconut oil is 1:4 to 4:1, preferably 1:1 to 3:1.

Preferably, the pH of the composition is below a pH of 5.0 such as a pH of from 4.0 to 5.0. Preferably, the pH of the composition is from 4.2 to 4.8. It is preferable to keep the pH of the composition within this range for compatibility with desired preservative systems. Preferably, the composition comprises sodium benzoate as a preservative.

Preferably, the composition has a viscosity of at least 3000 cPs, preferably at least 3100 cPs, preferably at least 3200 cPs. Preferably, the composition has a viscosity of no greater than 10,000 cPs, more preferably no greater than 8000 cPs, most preferably no greater than 6000 cPs.

Preferably the level of xanthan gum is from 0.1 to 2 wt % of the total composition, preferably 0.4 to 1.0 wt %

It is preferred if the product is a hair/scalp conditioning composition. Particularly preferred is a leave on treatment composition such as hair/scalp conditioner. In the context of the present invention the term leave on means the product is applied to the hair and is not rinsed off for at least a couple of hours, usually at least 10 hours.

Compositions of the invention may comprise a conventional anti-dandruff agent. Preferred antidandruff agents include Octopirox® (Piroctone olamine), climbazole and ketoconazole. Preferably, the anti-dandruff agent is in solution in the composition. The anti-dandruff agent is therefore preferably soluble in the composition of the invention at 25° C. Most preferably, the anti-dandruff agent is climbazole (1-imidazolyl-I-(4-chlorophenoxy)-3, 3-dimethylbutan-2-one). The anti-dandruff agent may be a single anti-dandruff compound or a mixture of different anti-dandruff compounds. If present preferably, the anti-dandruff agent is present in the composition in an amount of from 0.01 to 5% by total weight of the composition, more preferably from 0.1 to 5% by weight, most preferably 0.1 to 2% by total weight of the composition. Other scalp benefit agents for use in the present invention are, cholesterol and derivatives thereof, polyol esters, acetoglyceride esters, alkyl esters and alkenyl esters of fatty acids, lanolin and its derivatives, wax esters, beeswax and its derivatives, sterols, phospholipids, ceramides, neoceramides, pseudoceramides and mixtures thereof.

Also suitable for use in the present invention include vitamins, provitamins, derivatives thereof, and mixtures thereof. Especially preferred are B-vitamins and provitamins thereof, especially Vitamin B₃, panthenol and mixtures thereof. As used herein “Vitamin B₃” includes not only niacin (nicotinic acid), but also its corresponding amide (nicotinamide also known as niacinamide), as well as other amides and esters of niacin. The preferred form of Vitamin B₃ is niacinamide.

The invention will now be illustrated by the following non-limiting Examples. Examples of the invention are illustrated by a number, comparative Examples by a letter.

### EXAMPLES

Xanthan gum was dispersed in water and mixed and heated to 75° C. Montanov 68/Montanov S, were heated to 75-85° C. and added to the xanthan solution. The solution was cooled and preservatives were added followed by the pH adjuster under shear. Where applicable, pre-warmed remaining materials were then added under shear. The viscosity was measured on DHR Rheometer, between 16 and 24 hours after manufacture.

TABLE 1

| | | Example wt % | |
|---|---|---|---|
| INCI | Trade Name | A | B |
| Aqua | Water | To 100 | To 100 |
| Xanthan Gum | Xanthan Gum FNCS | 0.70 | 0.70 |
| Cetearyl Alcohol and | Montanov 68 | 2.25 | 2.25 |
| Cetearyl Glucoside | | | |
| 12-Hydroxystearic Acid | Casid HSA | 3.00 | — |
| Sodium Benzoate | Purox S | 0.50 | 0.50 |
| Citric Acid and Aqua | Citric Acid (50%) | 0.20 | 0.20 |
| Cocos Nucifera Oil | Agri Pure AP20 | — | 5.00 |

| | | |
|---|---|---|
| Viscosity (cPs) | 6156 | Not Stable |

Example B appeared grainy and was not stable, demonstrating that Montanov 68 is not a suitable thickener for compositions comprising coco nut oil without additional long chain fatty acids such as hydoxy stearic acid present.

TABLE 2

| | | Example wt % | | | |
|---|---|---|---|---|---|
| INCI | Trade Name | C | 1 | 2 | D |
| Aqua | Water | To 100 | To 100 | To 100 | To 100 |
| Xanthan Gum | Xanthan Gum | 0.70 | 0.70 | 0.70 | 0.70 |
| Coconut Alcohol and | Montanov S | 2.25 | 2.25 | 2.25 | 2.25 |
| Coco Glucoside | | | | | |
| 12-Hydroxystearic Acid | Casid HSA | 3.00 | — | 3.00 | 3.00 |
| Vegetable Stearic Acid | Emersol 7036 | — | — | — | 5.00 |
| Sodium Benzoate | Purox S | 0.50 | 0.50 | 0.50 | 0.50 |
| Citric Acid and Aqua | Citric Acid (50%) | 0.20 | 0.20 | 0.20 | 0.20 |
| Cocos Nucifera Oil | Agri Pure AP20 | — | 5.00 | 5.00 | — |

| | | | | |
|---|---|---|---|---|
| Viscosity (cPs) | 7828 | 3447 | 7998 | 12921 |

The above Examples demonstrate that Coconut Alcohol/Coco Glucoside is particularly effective in producing a composition with acceptable viscosity in the presence of Cocos Nucifera Oil (coconut oil), the presence of hydroxy stearic acid is additionally beneficial. When other oils are used such vegetable stearic acid an unacceptable viscosity is achieved.

TABLE 3

| | | Example wt % | | |
|---|---|---|---|---|
| INCI | Trade Name | E | 1 | F |
| Aqua | Water | To 100 | To 100 | To 100 |
| Xanthan Gum | Xanthan Gum | 0.70 | 0.70 | 0.70 |
| Coconut Alcohol and | Montanov S | 2.25 | 2.25 | 2.25 |
| Coco Glucoside | | | | |
| 12-Hydroxystearic Acid | Casid HSA | — | — | — |
| Vegetable Stearic Acid | Emersol 7036 | — | — | — |
| Sodium Benzoate | Purox S | 0.50 | 0.50 | 0.50 |
| Citric Acid and Aqua | Citric Acid (50%) | 0.20 | 0.20 | 0.20 |
| Cocos Nucifera Oil | Agri Pure AP20 | 11.25 | 5.00 | 0.45 |

| | | | |
|---|---|---|---|
| Viscosity (cPs) | 3980 | 3447 | 2728 |
| pH | 5.15 | 4.79 | 4.94 |

In the above table when the ratio of Coconut Alcohol/Coco Glucoside to Cocos Nucifera Oil (coconut oil) in the composition is below 1:4 (Example E) the pH of the composition is increased to above pH 5 which is not desirable for the operation of the preservative system.

When the ratio of Coconut Alcohol/Coco Glucoside to Cocos Nucifera Oil (coconut oil) is above 4:1 (Example F) a less desirable viscosity of below 3000 cPs is achieved.

1. A cosmetic composition comprising:
i) xanthan gum;
ii) a combination of a C₈ to C₁₄ alcohol and C₈ to C₁₄ glucoside; and
iii) from 0.5 to 10 wt % coconut oil;
wherein the weight ratio of ii) a combination of a C₈ to C₁₄ alcohol and C₈ to C₁₄ glucoside and iii) coconut oil is 1:4 to 4:1,
and wherein the pH of the composition is less than 5.0,
and wherein the viscosity of the composition is at least 3000 cPs.

2. (canceled)

3. The composition according to claim 1 in which the level of C₈ to C₁₄ alcohol and C₈ to C₁₄ glucoside ii) is from 0.5 to 7 wt % of the total composition.

4. The composition according to claim 1 which further comprises hydroxy stearic acid.

5. The composition according to claim 1 in which the weight ratio of ii) a combination of a C₈ to C₁₄ alcohol and C₈ to C₁₄ glucoside and iii) coconut oil is 1:1 to 3:1.

6. The composition according to claim 1 in which the level of xanthan gum is from 0.1 to 2 wt % of the total composition.

7. The composition according to claim 1 wherein the composition is a hair conditioning composition.

8. The composition according to claim 7 wherein the composition is a leave on hair conditioner.

9. A method of conditioning the hair and scalp comprising the step of applying to the hair and scalp the composition according to claim 1.

10. The composition according to claim 3, wherein the level of C₈ to C₁₄ alcohol and C₈ to C₁₄ glucoside ii) is from 1 to 5 wt % of the total composition.

11. The composition according to claim 4, further comprising 12-hydroxy stearic acid.

12. The composition according to claim 6, wherein the level of xanthan gum is 0.4 to 1.0 wt % of the total composition.
